(19) United States
(12) Patent Application Publication
(10) Pub. No.: US 2020/0316544 A1
(43) Pub. Date: Oct. 8, 2020

(54) QUATERNIZED ALKOXYLATED POLYMER SURFACTANT

(71) Applicants: Michael HEATH, Dallas, TX (US); ARC PRODUCTS, INC., Dallas, TX (US)

(72) Inventors: Michael HEATH, Dallas, TX (US); Bobby VITEAUX, Dallas, TX (US)

(73) Assignee: ARC PRODUCTS, INC., Dallas, TX (US)

(21) Appl. No.: 16/763,441

(22) PCT Filed: Dec. 18, 2018
(86) PCT No.: PCT/US18/66181

§ 371 Date: May 12, 2020

Related U.S. Application Data
(60) Provisional application No. 62/611,627, filed on Dec. 29, 2017.

(51) Int. Cl. B01F 17/00; C09K 8/60; C09K 8/68; C11D 1/00; C09D 7/45; C09D 7/65; A61Q 19/10; A61K 8/45; E21B 43/26

(52) U.S. Cl. CPC B01F 17/005; C09K 8/604; C09K 8/68; C11D 1/008; E21B 43/26; C09D 7/65; A61Q 19/10; A61K 8/45; C09D 7/45

(57) ABSTRACT

A quaternized alkoxylated polyethylene amine can be used in a variety of industries, including the oil and gas servicing industry, as a laundry detergent, the personal care industry, as an industrial cleaner, paint, or coating, and mining operations industry. A treatment fluid comprises: a base fluid; and the surfactant. A method of treating a subterranean formation comprises introducing the treatment fluid into a well, wherein the well penetrates the subterranean formation.

### TECHNICAL FIELD

Surfactants can be used in a variety of fluids in the oil and gas servicing industry. The surfactants can be cationic surfactants and impart desirable properties to the fluids.

### DETAILED DESCRIPTION

Oil and gas hydrocarbons are naturally occurring in some subterranean formations. In the oil and gas industry, a subterranean formation containing oil or gas is referred to as a reservoir. A reservoir may be located under land or off shore. Reservoirs are typically located in the range of a few hundred feet (shallow reservoirs) to a few tens of thousands of feet (ultra-deep reservoirs). In order to produce oil or gas, a wellbore is drilled into a reservoir or adjacent to a reservoir. The oil, gas, or water produced from the wellbore is called a reservoir fluid.

As used herein, a “fluid” is a substance having a continuous phase that tends to flow and to conform to the outline of its container when the substance is tested at a temperature of 71° F. (22° C.) and a pressure of 1 atmosphere (atm) (0.1 megapascals (MPa). Because of the nature and distribution of their natural hydrocarbon components, some reservoir “fluids” require temperatures higher than 71° F. to flow and to conform to the outlines of their containers. In such cases, testing and field treatments are often done at those higher temperatures. A fluid can be a liquid or gas. A homogenous fluid has only one phase, whereas a heterogeneous fluid has more than one distinct phase. A heterogeneous fluid can be: a slurry, which includes an external liquid phase and undissolved solid particles as the internal phase; an emulsion, which includes an external liquid phase and at least one internal phase of immiscible liquid droplets; a foam, which includes an external liquid phase and a gas as the internal phase; or a mist, which includes an external gas phase and liquid droplets as the internal phase. In some cases, heterogeneous reservoir fluids can be complex combinations of the above that may change with changes in variables such as temperature, pressure, and shear.

A well can include, without limitation, an oil, gas, or water production well, or an injection well. As used herein, a “well” includes at least one wellbore. A wellbore can include vertical, inclined, and horizontal portions, and it can be straight, curved, or branched. As used herein, the term “wellbore” includes any cased, and any uncased, open-hole portion of the wellbore. A near-wellbore region is the subterranean material and rock of the subterranean formation surrounding the wellbore. As used herein, a “well” also includes the near-wellbore region. The near-wellbore region is generally considered the region within approximately 100 feet radially of the wellbore. As used herein, “into a well” means and includes into any portion of the well, including into the wellbore or into the near-wellbore region via the wellbore.

A portion of a wellbore may be an open hole or cased hole. In an open-hole wellbore portion, a tubing string may be placed into the wellbore. The tubing string allows fluids to be introduced into or flowed from a remote portion of the wellbore. In a cased-hole wellbore portion, a casing is placed into the wellbore that can also contain a tubing string. A wellbore can contain an annulus. Examples of an annulus include, but are not limited to: the space between the wellbore and the outside of a tubing string in an open-hole wellbore; the space between the wellbore and the outside of a casing in a cased-hole wellbore; and the space between the inside of a casing and the outside of a tubing string in a cased-hole wellbore.

During wellbore operations, it is common to introduce a treatment fluid into the well. It is also common to introduce a treatment fluid into produced reservoir fluids above ground. A variety of treatment fluids are used in a variety of wellbore operations. Examples of common treatment fluids include, but are not limited to, drilling fluids, spacer fluids, cement compositions, completion fluids, work-over fluids, clean-up fluids, crude oil production, stimulation fluids, and storage and transportation of fluids. As used herein, a “treatment fluid” is a fluid designed and prepared to resolve a specific condition of a well or subterranean formation, such as for stimulation, isolation, gravel packing, or control of gas or water coning when used in the oil and gas servicing industry. The term “treatment fluid” refers to the specific composition of the fluid as it is being introduced into a well. The word “treatment” in the term “treatment fluid” does not necessarily imply any particular action by the fluid. When used in other industries, as used herein, the term “treatment fluid” means a fluid designed to achieve a desired result and provide specific properties, such as cleaning clothes, hair, skin, and other surfaces, and paint formulations. By way of example, some desired results for a paint formulation can include defoaming, better dispersion of pigments, better adhesion to surfaces, improved leveling and flow properties, among others.

Hydraulic fracturing, sometimes simply referred to as “fracturing” or “fracing,” is a common stimulation treatment. A treatment fluid adapted for this purpose is sometimes referred to as a fracturing fluid or “frac fluid.” The fracturing fluid is pumped at a sufficiently high flow rate and high pressure into the wellbore and into the subterranean formation to create a fracture in the subterranean formation. As used herein, “creating a fracture” means making a new fracture in the formation or enlarging a pre-existing fracture in the formation. The fracturing fluid may be pumped down into the wellbore at high rates and pressures, for example, at a flow rate in excess of 100 barrels per minute (3,150 U.S. gallons per minute) at a pressure in excess of 5,000 pounds per square inch (“psi”) (35 megapascals “MPa”).

Additionally, some treatment fluids are used in above ground operations to bring about desired effects, such as dehydration, desalination, and clean phase separation of undesirable components. The treatment fluids generally contain a base fluid and one or more additives. As used herein, the term “base fluid” means the liquid that is in the greatest concentration and is the solvent of a solution or the continuous phase of a heterogeneous fluid.

Additional applications of treatment fluids include, but are not limited to, augmenting the dehydration and clean separation of oil and water-phases indigenous to produced hydrocarbon liquids, to help break and prevent formation of emulsions during subterranean flow, to impart differential wetting of subterranean surfaces to facilitate concurrent flow of liquids, to disperse problematic colloidal solids and heavy hydrocarbons, to augment the inhibition of water imbibition, hydration and swelling of water-sensitive subterranean rock formations, and to facilitate the removal of undesirable materials from surfaces. Other additional applications of a treatment fluid include detergents (e.g., for clothes), personal care formulations (e.g., hair shampoos and conditioners, hand soaps), industrial cleaners, paints and coatings, and mining operations. There may be other industrial applications not specifically mentioned that the disclosed surfactant and treatment fluid containing the surfactant may be used in.

A surfactant is one type of additive that can be included in a treatment fluid. The surfactant can impart desirable properties to the treatment fluid. A surfactant is an amphiphilic molecule comprising a hydrophobic tail group and a hydrophilic head group. The hydrophilic head can be charged. A cationic surfactant includes a positively-charged head. An anionic surfactant includes a negatively-charged head. A zwitterionic surfactant includes both a positively- and negatively-charged head. A surfactant with no charge is called a non-ionic surfactant.

A surfactant can lower the interfacial tension between two liquids or between a solid and a liquid. As such, a surfactant can be used to reduce the surface tension between the solids of a subterranean formation and the treatment fluid in order for the treatment fluid to penetrate farther into the formation. A surfactant can also be used to change the wettability of the surface of solids of a formation. Wettability means the preference of a surface to be in contact with one liquid or gas rather than another. Accordingly, “oil-wet” means the preference of a surface to be in contact with an oil phase or gas phase rather than a water phase, and “water-wet” means the preference of a surface to be in contact with a water phase rather than an oil phase or gas phase. A surfactant can be used to change the wettability of the surface of the solids from being water-wet to being oil-wet or vice versa. In some cases, surfactants adsorbed onto a surface can equalize or even lessen the affinity of both oil and water to that surface. Such wettability changes can help promote production of oil and/or gas from a reservoir.

If a surfactant is in a sufficient concentration in a solution, then the surfactant molecules can form micelles. A “micelle” is an aggregate of surfactant molecules dispersed in a solution. A surfactant in an aqueous solution can form micelles with the hydrophilic heads in contact with the surrounding aqueous solvent, sequestering the hydrophobic tails in the micelle center. The surfactant must be in a sufficient concentration to form micelles, known as the critical micelle concentration (CMC). The critical micelle concentration is the concentration of surfactant above which micelles are spontaneously formed. Some surfactant functions, such as those that involve lowering surface tension, are optimized by the surfactant being at or above its CMC in the bulk phase; whereas other interfacial surfactant functions, such as those involved in emulsion breaking or prevention, are optimized at concentrations well below the CMC.

There is an ongoing industry-wide search for new surfactants that can be used more effectively in treatment fluids.

It has been discovered that a quaternized alkoxylated polyethylene amine (PEA) polymer cationic surfactant can be used in treatment fluids. One of the advantages to the new surfactant is improved properties to the treatment fluid. The treatment fluid can be used in the following industries by way of non-limiting examples: the oil and gas servicing industry, detergents (e.g., for clothes), personal care formulations (e.g., hair shampoos and conditioners, and hand soaps), industrial cleaners, paints and coatings, and mining operations.

A polymer is a large molecule composed of repeating units, typically connected by covalent chemical bonds. A polymer is formed from monomers. During the formation of the polymer, some chemical groups can be lost from each monomer. The piece of the monomer that is incorporated into the polymer is known as the repeating unit or monomer residue. The backbone of the polymer is the continuous link between the monomer residues. The polymer can also contain functional groups connected to the backbone at various locations along the backbone. Polymer nomenclature is generally based upon the type of monomer residues comprising the polymer. A polymer formed from one type of monomer residue is called a homopolymer. A copolymer is formed from two or more different types of monomer residues. The number of repeating units of a polymer is referred to as the chain length of the polymer. The number of repeating units of a polymer can range from approximately 11 to greater than 10,000. In a copolymer, the repeating units from each of the monomer residues can be arranged in various manners along the polymer chain. For example, the repeating units can be random, alternating, periodic, or block. The conditions of the polymerization reaction can be adjusted to help control the average number of repeating units (the average chain length) of the polymer.

A polymer has an average molecular weight, which is directly related to the average chain length of the polymer. The average molecular weight of a polymer has an impact on some of the physical characteristics of a polymer, for example, its solubility and its dispersibility. For a copolymer, each of the monomers will be repeated a certain number of times (number of repeating units). The average molecular weight (M_{w}) for a copolymer can be expressed as follows:

$${M}_{w} = \sum {w}_{x} {M}_{x}$$

where wₓ is the weight fraction of molecules whose weight is Mₓ.

According to an embodiment, a treatment fluid comprises: a base fluid; and a surfactant, wherein the surfactant is a quaternized alkoxylated polyethylene amine.

According to another embodiment, a method of treating a portion of a subterranean formation comprises: introducing the treatment fluid into a well, wherein the well penetrates the subterranean formation.

The discussion of preferred embodiments regarding the treatment fluid or any ingredient in the treatment fluid, is intended to apply to the composition embodiments and the method embodiments. Any reference to the unit “gallons” means U.S. gallons.

The treatment fluid can be a homogenous fluid or a heterogeneous fluid. The treatment fluid can be a slurry, emulsion, or invert emulsion. The treatment fluid includes a base fluid. The base fluid can include water. The water can be selected from the group consisting of freshwater, brackish water, saltwater, and any combination thereof. The base fluid can further include a water-soluble salt. Preferably, the salt is selected from the group consisting of sodium chloride, calcium chloride, calcium bromide, potassium chloride, potassium bromide, potassium formate, magnesium chloride, sodium bromide, cesium formate, cesium acetate, and any combination thereof.

The base fluid can also include a hydrocarbon liquid. As used herein, the phrase “hydrocarbon liquid” means a pure hydrocarbon liquid or a hydrocarbon-containing liquid. The hydrocarbon liquid can be selected from the group consisting of: a fractional distillate of crude oil; a fatty derivative of an acid, an ester, an ether, an alcohol, an amine, an amide, or an imide; a saturated hydrocarbon; an unsaturated hydrocarbon; a branched hydrocarbon; a cyclic hydrocarbon; and any combination thereof. Crude oil can be separated into fractional distillates based on the boiling point of the fractions in the crude oil. An example of a suitable fractional distillate of crude oil is diesel oil. The saturated hydrocarbon can be an alkane or paraffin. Preferably, the saturated hydrocarbon is an alkane. The paraffin can be an isoalkane (isoparaffin), a linear alkane (paraffin), or a cyclic alkane (cycloparaffin). The unsaturated hydrocarbon can be an alkene, alkyne, or aromatic. The alkene can be an isoalkene, linear alkene, or cyclic alkene. The linear alkene can be a linear alpha olefin or an internal olefin.

The treatment fluid can be a variety of different types of fluids and be used in a variety of different types of oilfield operations, such as at a wellsite, in transportation and storage of liquid hydrocarbons, and in refineries. Non-limiting examples of uses of the surfactant additive include in a wellbore fluid, as a non-emulsifier, as an emulsion breaker or de-emulsifier, wetting-out and dispersing asphaltenes in crude oils, de-salting of refinery fluids (i.e., washing residual salts out of the crude oil with fresh water before refining), as a macro-emulsifier or micro-emulsifier, and adsorbing onto subterranean rock surfaces to positively impact the out-flow of indigenous fluids and stabilize those subterranean surfaces by discouraging imbibition of damaging fresher waters.

The treatment fluid includes the surfactant. The surfactant can be a cationic surfactant. The surfactant can be a quaternized alkoxylated polyethylene amine (PEA) polymer. Alkoxylation is a chemical reaction that involves the addition of an epoxide to another compound. It is to be understood that all compounds that are ethoxylated are also considered to be alkoxylated; however, not all alkoxylated compounds are also inherently ethoxylated. According to certain embodiments, the alkoxylated surfactant is not considered to be ethoxylated. Additionally, according to certain embodiments, the nature of the alkoxylating agent(s), as well as the degree(s) and sequence(s) of alkoxylation can vary.

According to certain embodiments, the PEA polymer is diethylene triamine (DETA), triethylene tetramine (TETA) or tetraethylene pentamine (TEPA). The molecular weight of the PEA polymer can vary. The PEA polymer can have a molecular weight greater than about 100. The PEA polymer can also have a molecular weight in the range of about 100 to about 1,000,000, preferably about 100 to about 100,000, more preferably about 100 to about 10,000. The PEA polymer can also have a molecular weight such that the surfactant is soluble or dispersible in the base fluid. As used herein, the term “soluble” means that at least one part of the substance dissolves in 10,000 parts of a liquid.

The surfactant is quaternized. A quaternary compound is a cation consisting of a central positively charged atom with four substituents, especially organic (alkyl and aryl) groups, discounting hydrogen atoms. The number of nitrogen atoms of the surfactant that are quaternized can vary. By way of example, the number of nitrogen atoms that are quaternized can range from 1 to 5. It is to be understood that the compound that is quaternized can include from 3 to 5 nitrogen atoms. An example of a compound including: 3 nitrogen atoms is diethylene triamine (DETA); 4 nitrogen atoms is triethylene tetramine (TETA); and 5 nitrogen atoms is tetraethylene pentamine (TEPA). It is to also be understood that even if the compound includes 5 nitrogen atoms, that not all of the nitrogen atoms need to be quaternized. The degree of quaternization can be controlled and selected based on the desired properties of the surfactant.

The agents used to quaternize the surfactant can vary. Depending upon the surfactant properties desired in a particular treatment fluid, the agent(s) used to quaternize the surfactant can vary, which affects (1) which hydrocarbon group becomes the fourth to attach to the nitrogen(s) in quaternization and 2) what the surfactant counter-anion(s) will be. Non-limiting variations include methyl-, ethyl-, or benzyl-quaternization agents with Cl⁻, Br⁻, or SO₄⁼ counter-anion(s).

The following is but one, non-limiting, example of a surfactant according to certain embodiments.

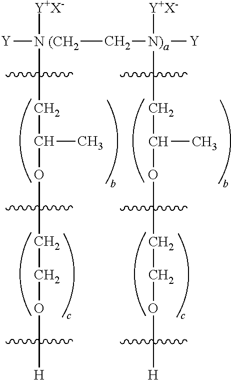

where Y=methyl, ethyl, benzyl, etc.; X=Cl⁻, Br⁻, I⁻, or ½SO₄⁼; a=2−4; b=1−100; and c=0−40.

The following is but one, non-limiting, example of a reaction sequence for preparation of the surfactant according to certain embodiments.

tetraethylene pentamine (TEPA)+propylene oxide (PO)+ethylene oxide (EO)→TEPA alkoxylate+methyl chloride→quaternized TEPA alkoxylate.

The concentration of the TEPA can be in the range from about 0.2% to about 10%; the concentration of the propylene oxide in the range of about 30% to about 97%; the concentration of the ethylene oxide in the range of about 3% to about 50% by weight of the compounds reacting to form the TEPA alkoxylate. The compounds reacting to form the TEPA alkoxylate can further include potassium hydroxide in a concentration in the range of about 0.01% to about 1%. The TEPA alkoxylate can be represented by the following structure:

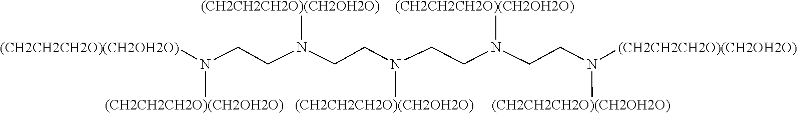

The alkoxylated PEA polymer can then be reacted with a quaternization agent containing methyl, ethyl, or benzyl to quaternize the nitrogen atoms in the polymer. By way of but one non-limiting example, the alkoxylated PEA polymer can be reacted with methyl chloride. As discussed above, the reaction conditions can be adjusted and controlled to provide a desired amount of quaternization. The alkoxylated PEA polymer can be reacted at a concentration in the range of about 95% to about 99.9% by weight with the quaternization agent in a concentration of about 0.25% to about 5% by weight. A higher concentration of the quaternization agent can cause more of the nitrogen atoms of the alkoxylated PEA polymer to become quaternized. A fully quaternized TEPA alkoxylate that has been reacted with methyl chloride can be represented by the following chemical structure:

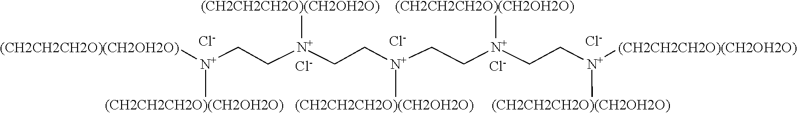

The surfactant can be included in the base fluid in a concentration in the range of about 0.0001% to about 40% by weight of the base fluid.

The treatment fluid can also contain various other additives and have a variety of desirable properties based on the type of treatment fluid and industry uses. The other additives can be selected based on the type of treatment fluid. By way of one example, if the treatment fluid is a frac fluid, then the fluid can also include proppant, a viscosifier, etc. Other additives can include cement, proppant, a viscosifier, a suspending agent, a weighting agent, a friction reducer, a filler, a fluid loss additive, a set retarder, a strength-retrogression additive, a light-weight additive, a defoaming agent, a mechanical property enhancing additive, a lost-circulation material, a filtration-control additive, a thixotropic additive, and combinations thereof. The other additives can also be selected based on the type of industry the treatment fluid is used in. One of ordinary skill in the art will be able to select the other additives and concentration based on the industry. By way of example, for a detergent for cleaning clothes, the treatment fluid can further include one or more anionic or non-ionic surfactants, one or more solvents, chelating agents, suspending agents, bleach, de-foaming agents, foaming agents, fragrances, process aids, anti-redeposition agents, and enzymes.

The methods include introducing the treatment fluid into a well, wherein the well penetrates the subterranean formation. The well can be an oil, gas, or water production well, a geothermal well, or an injection well. The well can include a wellbore. The subterranean formation can be part of a reservoir or adjacent to a reservoir. The step of introducing the treatment fluid can be for the purpose of: drilling a wellbore using a drilling fluid treatment; cementing a portion of the wellbore using a cement composition; flushing a drilling fluid from the wellbore prior to introduction of a cement composition using a spacer fluid; or creating fractures within the subterranean formation. The treatment fluid can be in a pumpable state before and during introduction into the well. The treatment fluid can be mixed prior to introduction. The step of mixing can include using a mixing apparatus. The treatment fluid can also be introduced into the well using a pump.

The exemplary fluids and additives disclosed herein may directly or indirectly affect one or more components or pieces of equipment associated with the preparation, delivery, recapture, recycling, reuse, and/or disposal of the disclosed fluids and additives. For example, the disclosed fluids and additives may directly or indirectly affect one or more mixers, related mixing equipment, mud pits, storage facilities or units, fluid separators, heat exchangers, sensors, gauges, pumps, compressors, and the like used generate, store, monitor, regulate, and/or recondition the exemplary fluids and additives. The disclosed fluids and additives may also directly or indirectly affect any transport or delivery equipment used to convey the fluids and additives to a well site or downhole such as, for example, any transport vessels, conduits, pipelines, trucks, tubulars, and/or pipes used to fluidically move the fluids and additives from one location to another, any pumps, compressors, or motors (e.g., topside or downhole) used to drive the fluids and additives into motion, any valves or related joints used to regulate the pressure or flow rate of the fluids, and any sensors (i.e., pressure and temperature), gauges, and/or combinations thereof, and the like. The disclosed fluids and additives may also directly or indirectly affect the various downhole equipment and tools that may come into contact with the fluids and additives such as, but not limited to, drill string, coiled tubing, drill pipe, drill collars, mud motors, downhole motors and/or pumps, floats, MWD/LWD tools and related telemetry equipment, drill bits (including roller cone, PDC, natural diamond, hole openers, reamers, and coring bits), sensors or distributed sensors, downhole heat exchangers, valves and corresponding actuation devices, tool seals, packers and other wellbore isolation devices or components, and the like.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is, therefore, evident that the particular illustrative embodiments disclosed above may be altered or modified and all such variations are considered within the scope and spirit of the present invention.

As used herein, the words “comprise,” “have,” “include,” and all grammatical variations thereof are each intended to have an open, non-limiting meaning that does not exclude additional elements or steps. While compositions, systems, and methods are described in terms of “comprising,” “containing,” or “including” various components or steps, the compositions, systems, and methods also can “consist essentially of” or “consist of” the various components and steps. It should also be understood that, as used herein, “first,” “second,” and “third,” are assigned arbitrarily and are merely intended to differentiate between two or more atoms, etc., as the case may be, and does not indicate any sequence. Furthermore, it is to be understood that the mere use of the word “first” does not require that there be any “second,” and the mere use of the word “second” does not require that there be any “third,” etc.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent(s) or other documents that may be incorporated herein by reference, the definitions that are consistent with this specification should be adopted.

What is claimed is:

1. A treatment fluid comprising:
a base fluid; and
a surfactant, wherein the surfactant is a quaternized alkoxylated polyethylene amine.

2. The treatment fluid according to claim 1, wherein the base fluid comprises water.

3. The treatment fluid according to claim 1, wherein the base fluid comprises a hydrocarbon liquid.

4. The treatment fluid according to claim 3, wherein the hydrocarbon liquid is selected from the group consisting of: a fractional distillate of crude oil; a fatty derivative of an acid, an ester, an ether, an alcohol, an amine, an amide, or an imide; a saturated hydrocarbon; an unsaturated hydrocarbon; a branched hydrocarbon; a cyclic hydrocarbon; and any combination thereof.

5. The treatment fluid according to claim 1, wherein the surfactant is a cationic surfactant.

6. The treatment fluid according to claim 1, wherein the polyethylene amine is selected from diethylene triamine, triethylene tetramine, or tetraethylene pentamine.

7. The treatment fluid according to claim 1, wherein the polyethylene amine has a molecular weight in the range of about about 100 to about 10,000.

8. The treatment fluid according to claim 1, wherein the polyethylene amine is alkoxylated by reacting the polyethylene amine with at least one of propylene oxide and ethylene oxide.

9. The treatment fluid according to claim 8, wherein at least one of the nitrogen atoms of the alkoxylated polyethylene amine is quaternized by reacting the alkoxylated polyethylene amine with a quaternization agent.

10. The treatment fluid according to claim 9, wherein the number of nitrogen atoms that are quaternized range from 1 to 5.

11. The treatment fluid according to claim 9, wherein the quaternization agent is selected from compounds comprising methyl-, ethyl-, or benzyl- substituents with Cl⁻, Br⁻, or SO₄⁻² counter-anions.

12. The treatment fluid according to claim 11, wherein the alkoxylated polyethylene amine is in a concentration in the range of about 95 to about 99.9 weight percent and the quaternization agent is in a concentration in the range of about 0.25 to about 5 weight percent.

13. The treatment fluid according to claim 1, wherein the surfactant is in a concentration in the range of about 0.0001% to about 40% by weight of the base fluid.

14. The treatment fluid according to claim 1, wherein the treatment fluid is an oil and gas servicing treatment fluid.

15. The treatment fluid according to claim 14, wherein the treatment fluid further comprises proppant, a viscosifier, cement, a suspending agent, a weighting agent, a friction reducer, a filler, a fluid loss additive, a set retarder, a strength-retrogression additive, a light-weight additive, a defoaming agent, a mechanical property enhancing additive, a lost-circulation material, a filtration-control additive, a thixotropic additive, and combinations thereof.

16. The treatment fluid according to claim 1, wherein the treatment fluid is a laundry detergent, a personal care formulation, an industrial cleaner, a paint or coating, or a mining operation fluid.

17. A surfactant comprising:
a quaternized alkoxylated polyethylene amine.

18. A method of treating a subterranean formation comprising:
introducing a treatment fluid into a well, wherein the well penetrates the subterranean formation, and wherein the treatment fluid comprises:
a base fluid; and
a surfactant, wherein the surfactant is a quaternized alkoxylated polyethylene amine.

19. The method according to claim 18, wherein the treatment fluid is a stimulation fluid.

20. The method according to claim 19, further comprising creating one or more fractures within the subterranean formation during the step of introducing the treatment fluid into the well.
